(19) United States
(12) Plant Patent Application Publication
(10) Pub. No.: US 2019/0191610 P1
(43) Pub. Date: Jun. 20, 2019

(54) Apple tree named 'RDS'

(50) ;

(71) Applicants: Ashley Green, Lenswood (AU); Brenton Green, Lenswood (AU)

(72) Inventors: Ashley Green, Lenswood (AU); Brenton Green, Lenswood (AU)

(73) Assignee: Fruit Varieties International Pty Ltd., Grove (AU)

(21) Appl. No.: 16/350,662

(22) Filed: Dec. 17, 2018

(30) Foreign Application Priority Data
Dec. 18, 2017 (AU) AU 2017/313

(51) Int. Cl. A01H 6/74

(52) U.S. Cl. PLT/161

(57) ABSTRACT

A new and distinct apple tree Malus domestica named ‘RDS.’ ‘RDS’ is a spontaneous limb mutation of the variety ‘Cripps Red’ and is distinguished by its early color development, a reddish brown current season wood growth on the sunny side, and a high level of red anthocyanin on the back of the leaf petiole and on young fruitlets. It is further distinguished by its strong bloom on fruit at maturity, a very dark intensity purple red hue of overcolor and a narrow stripe with solid flush having weakly defined stripes overcolor.

Botanical classification: Malus domestica.

Variety denomination: The new apple tree variety denomination is ‘RDS’.

### BACKGROUND OF THE VARIETY

The present invention is a new and distinct cultivar of apple tree botanically known as Malus domestica and referred to by the cultivar name ‘RDS’. ‘RDS’ was discovered as a spontaneous limb mutation in a commercial orchard of the variety ‘Cripps Red’ (U.S. Plant Pat. No. 8,477).

‘RDS’ was first observed in 2013 in Lenswood, South Australia, Australia, where it was distinguished by its dark purple red fruit color. ‘RDS’ is further distinguished from its parent by its early color development: reddish brown current season wood growth on the sunny side with a high level of red anthocyanin on the back of the leaf petiole and a high level of red anthocyanin on young fruitlets. It is further distinguished by a strong bloom on fruit at maturity, a very dark intensity purple red hue overcolor, and a narrow stripe with solid flush overcolor. Asexual reproduction of ‘RDS’ was carried out by bud grafting in 2014 and trees of ‘RDS’ were planted for additional observation at Lenswood, South Australia, Australia. ‘RDS’ has since been fruited through successive asexually propagated generations at Lenswood, South Australia and has been observed to remain true to type with the distinguishing characteristics retained through successive generations of asexual reproduction.

### BRIEF DESCRIPTION OF THE VARIETY

‘RDS’ is primarily distinguished by its dark purple red fruit color. ‘RDS’ is also distinguished from its parent by its early color development: a reddish brown current season wood growth on the sunny side, a high level of red anthocyanin on the back of the leaf petiole, and a high level of red anthocyanin on young fruitlets. ‘RDS’ also exhibits a strong bloom on fruit at maturity, a very dark intensity purple red hue overcolor and a narrow stripe with solid flush having weakly defined stripes overcolor. These and other distinguishing characteristics are set forth in the tables below. Under similar growing conditions in Tasmania, Australia, under commercial practice, ‘RDS’ is distinguishable from its parent ‘Cripps Red’ as described in Table 1 below:

TABLE 1
Comparison of ‘RDS’ to ‘Cripps Red’

| Characteristic | ‘Cripps Red’ | ‘RDS’ |
|---|---|---|
| Fruit: Relative Area of | Medium to Large | Very Large |
| Overcolor | | |
| Fruit: Hue of Overcolor with | Red-purple 60A | Red-purple 59A |
| Bloom removed | | |
| Fruit: Intensity of Overcolor | Light to Medium | Dark |
| Fruit: Width of Stripes | Medium to broad | Very narrow to Narrow |
| Young Fruit: Extent of | Very small to small—21% on | Large—61% on average |
| Anthocyanin Over color | average of fruitlet covered with | of fruitlet covered with |
| | Anthocyanin | Anthocyanin |
| Petiole: Extent of | Small—4.40 mm on average | Large—21.90 mm on |
| Anthocyanin coloration from | | average |
| base | | |
| 1 Year Old Shoot: | Medium-brown 175A | Reddish-brown N186C |
| Sun Exposed Side Color | | |
| 1 Year Old Shoot: | Medium—95 on average per 100 | Very few to few—78 on |
| Number of Lenticels | mm of stem | average per 100 mm of |
| | | stem |

### BRIEF DESCRIPTION OF THE PHOTOGRAPHS

Typical specimens of the tree and blossoms for the new apple tree variety ‘RDS’ of a 3-year-old tree planted on ‘MM106’ rootstock (unpatented) are shown in the accompanying photographs. The colors shown are as true as possible within the usual limits of this kind of illustration.

FIG. 1 shows current season's fresh fruit of ‘RDS’ harvested at maturity;

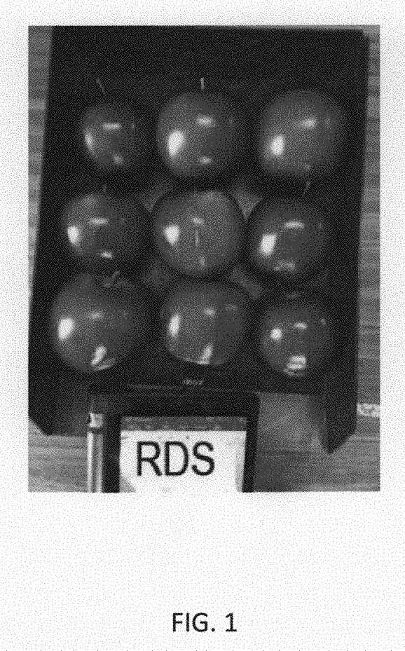

FIG. 2 shows current season's fresh fruit of ‘RDS’ (on the left) compared to fruit of ‘Cripps Red’ (on the right), both harvested at maturity from trees of similar age and growing conditions;

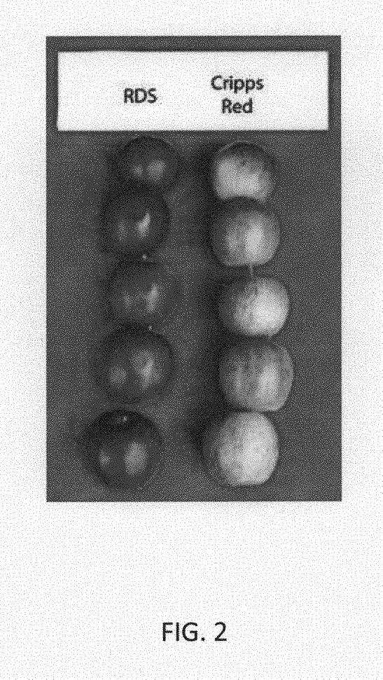

FIG. 3 shows the parent tree ‘Cripps Red’ and early color development of ‘RDS’ limb sport;

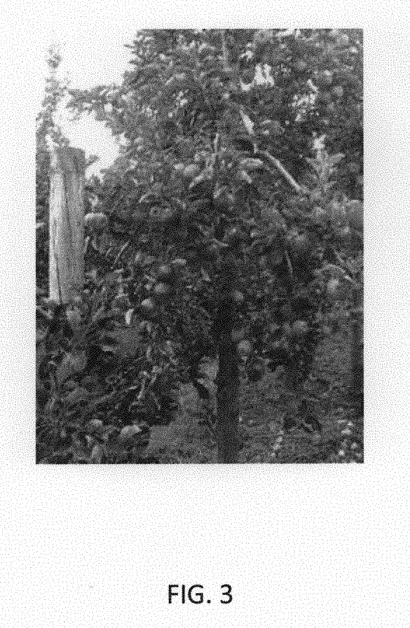

FIG. 4 shows the under-side of leaves of a 3-year-old current season's shoot of ‘RDS’ in its third growing season on ‘MM106’ rootstock;

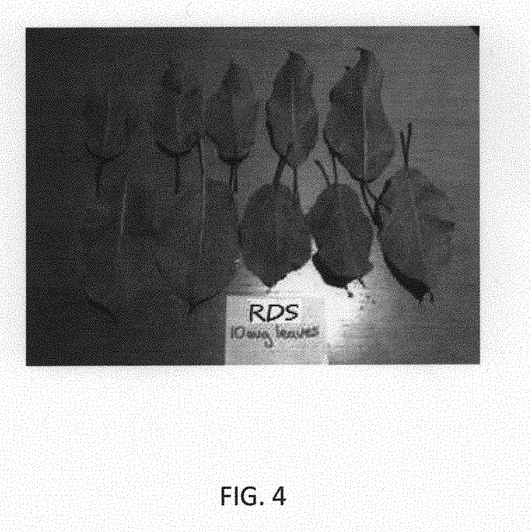

FIG. 5 shows the upper-side of leaves of a 3-year-old current season's shoot of ‘RDS’ in its third growing season on ‘MM106’ rootstock;

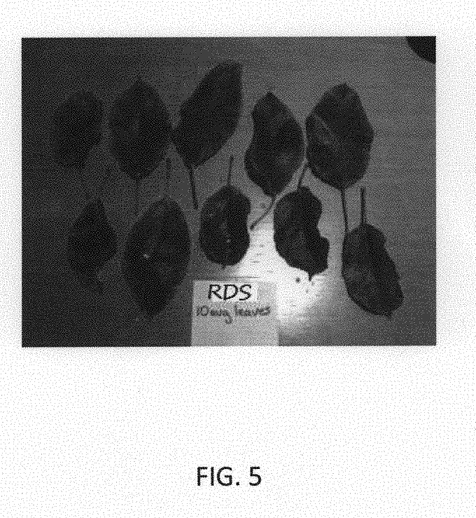

FIG. 6 shows a blossom of ‘RDS’ on an ‘RDS’ tree in its third growing season on ‘MM106’ rootstock; and,

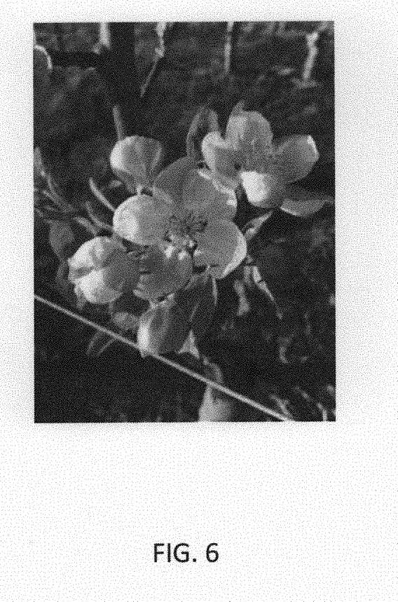

FIG. 7 shows the fruit of ‘RDS’ on an ‘RDS’ tree in its third growing season on ‘MM106’ rootstock.

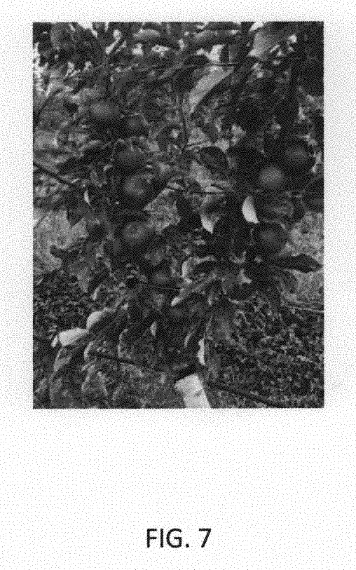

### DETAILED BOTANICAL DESCRIPTION OF THE VARIETY

The following-detailed botanical description is based on observations made during the third growing season at Lucaston, Tasmania, Australia of 3-year-old trees planted on ‘MM106’ rootstock. The cultivar has not been observed under all possible environmental conditions. The phenotype may vary significantly with variations in the environment such as temperature, length of day and light intensity, without any variance in genotype. It should be understood that the characteristics described will vary somewhat depending upon cultural practices and climatic conditions, and will vary with location and season. The plant and flower quantified measurements and foliage size are expressed as an average of measurements taken from several individual plants of ‘RDS’ using standard commercial cultural practices. The measurements of any individual plant or any group of plants of ‘RDS’ may vary from the stated average. All colors are described according to The Royal Horticultural Society Colour Chart (5^{TH} edition 2007).
Tree:
   Vigor.—Medium.
   Type.—Ramified — Bearing on Spurs and Long Shoots.
   Habit.—Spreading.
   Height.—3.6 m.
   Trunk diameter.—45 mm at 100 mm above the graft.
   Bark texture.—Smooth.
   Bark color.—Greyed-red on sunny side 181A.
   Branch.—Fruiting branches located at around 1 m above the graft union.
   Length.—1.5 m average.
   Diameter.—18 mm at base.
   Crotch angle.—Branches on unpruned tree are horizontal.
   Bark color.—Greyed-red 181A on sun exposed side.
   Lenticel length.—0.8 mm to 1.2 mm Average is 1 mm.
   Lenticel color.—Orange-white 159A.
   Quantity of lenticels per cm².—3 to 7 per cm².
One year old shoot:
   Length.—205 mm average.
   Color.—Greyed-purple 186C.
   Pubescence.—Weak.
   Thickness.—6.5 mm average.
   Internode length.—13.3 mm average.
   Number of lenticels per cm².—4 to 8 per cm²on average.
Flowers:
   Diameter of fully open flower.—36 mm on average.
   Relative position of petal margin.—Touching to occasionally free.
   Quantity of flowers per cluster.—5 to 7.
   Date of beginning of flowering.—24^{th} of September in Tasmania.
   Date of full bloom.—30^{th} of September in Tasmania.
   Pollination requirement.—Pollination required by another diploid variety flowering at similar time.
Petals:
   Quantity per flower.—5.
   Shape.—Ovate to ellipsoid.
   Length.—23 mm.
   Width.—15.8 mm.
   Apex.—Ovoid.
   Base.—Obtuse.
   Margin.—Smooth.
   Color—upper surface.—Red-purple RHS 64D.
   Color—lower surface.—Red-purple RHS 63C.
   Pistils.—7.7 mm long; Yellow-green RHS 144C Color.
   Stigma.—0.5 mm diameter; Yellow-green RHS 144C Color.
   Position of stigma relative to anther.—Same level.
   Style.—5 fused at base; Length 6.25 mm; Color is 145C Yellow-green; Pubescent at base.
   Ovary.—Pubescent; 1.51 mm average diameter; Color Yellow-green 144C.
   Anthers.—19 mm average per flower; Length 2.3 mm; Width 1.9 mm; Ample pollen quantity, 10C Yellow Color.
   Pedicel.—Length 16.5 mm average; Diameter 1.5 mm; Color: 187C Grey-purple on sun exposed side; 144B Yellow-green on shaded side.
   Sepals.—Quantity 5; Length 5.8 mm average; Color: 59B Red-purple at base with 144B Yellow-green from base to tip with smooth straight margin and pubescent.
Leaves:
   Length.—113 mm average.
   Width.—70 mm average.
   Length/width ratio.—1.6:1.
   Blade margin.—Serrate Type 1.
   Shape.—Oval to obtuse.
   Apex.—Acute in shape.
   Base.—Obtuse in shape.
   Pubescence.—Description: Not pubescent on underside.
   Color—upper surface.—Green N137D.
   Color—lower surface.—Green N146B.
   Attitude in relation to shoot.—Outward.
   Petiole.—Length 39.5 mm average; Diameter 2.5 mm average; Color: Grey-purple 184A.
Fruit:
   Quantity per cluster.—5 to 7 per cluster if no thinning occurs.
   Diameter.—76.5 mm average.
   Height.—69.3 mm average.
   Ratio of height to width.—About 1:0.91.
   General shape in profile.—Globose.
   Position of maximum diameter.—Near center.
   Ribbing.—Moderate.
   Crowning at calyx end.—Absent or weak.
   Size of eye.—9.6 mm average.
   Aperture of eye.—Mostly closed, occasionally open.
   Length of sepal.—5.2 mm average length.
   Bloom of skin.—Moderate to strong.
   Greasiness of skin.—Absent or weak.
   Background color of skin.—Yellow-green 154D.
   Amount of over color.—Very large.
   Over color of skin.—Red-purple 59A.
   Intensity of over color.—Dark.
   Pattern of over color.—Solid Flush with weakly defined stripes.
   Amount of russet around stalk cavity.—Medium to low.
   Amount of russet on cheeks.—Absent.
   Area of russet around eye basin.—Absent.
   Length of stalk.—24.5 mm average.
   Thickness of stalk.—2.1 mm average.
   Stalk color.—Greyed-orange 165B.
   Depth of stalk cavity.—12.1 mm average.
   Width of stalk cavity.—30.9 mm average.
   Depth of eye basin.—5.2 mm average.
   Width of eye basin.—29.1 mm average.
   Flesh color.—White 155A.
   Firmness of flesh.—8.3 kg, Firm.
   Flesh texture.—Firm and crisp.
   Aroma.—Fresh.
   Juiciness.—Ample.
   Total soluble solids.—15° Brix.
   Seeds.—Minimum of 1, mostly 2 per cell; 8 per fruit; Acute; Greyed-orange 166B; Aperture of locules closed or slightly open.
Harvest:
   Harvest date.—Time of eating maturity.—Mid to Late season, Apr. 26, 2018 in Tasmania, Australia.
   Number of picks.—One.
   Yield.—21 kg from typical three-year-old tree.
Disease/insect resistance/susceptibility: No resistance/susceptibility noted Winter hardiness, cold tolerance, drought, and heat tolerance are similar to parent variety. The fruit coloration is retained in warmer environments.
Market use: Fresh.

We claim:

1. A new and distinct apple tree substantially as described and illustrated herein.
